(19) United States
(12) Patent Application Publication
(10) Pub. No.: US 2016/0073943 A1
(43) Pub. Date: Mar. 17, 2016

(54) Lancing Device with Improved Lancet Holder

(71) Applicant: BIOTEST MEDICAL CORPORATION, Taichung City (TW)

(72) Inventor: Chen-Heng CHENG, Taichung City (TW)

(21) Appl. No.: 14/852,105

(22) Filed: Sep. 11, 2015

(30) Foreign Application Priority Data
Sep. 11, 2014 (TW) 103216165

(51) Int. Cl. A61B 5/151

(52) U.S. Cl. CPC A61B 5/15132; A61B 5/15186

(57) ABSTRACT

A lancing device includes a front cap detachably capped on a front side of a shell, a lancet, and a lancet holder linearly movably mounted in the shell for securing the lancet. The lancet holder is configured to provide one or multiple guide portions at the outer perimeter thereof. The front cap has one or multiple guide tracks located at an inner wall thereof corresponding to the stroke of the guide portion of the lancet holder. Thus, under the guidance of the guide track, the lancet and the lancet holder can be moved smoothly with minimized vibration to reduce the sense of pain suffered by the user.

### BACKGROUND OF THE INVENTION

1. Field of the Invention

The present invention relates to human blood sampling device, and more particularly to a lancing device, which is equipped with an improved lancet holder that holds the lancet firmly in position and moves the lancet smoothly with least vibration to reduce the sense of pain suffered by the user.

2. Description of the Related Art

Lancing devices are most commonly used by diabetics or patients with chronic diseases during blood glucose monitoring. In application, lancets are used to make punctures, such as a fingerstick, to obtain small blood specimens.

A conventional lancing device generally comprises a pen-like shell, a front cap capped on the front end of the shell, a lancet detachably mounted in a lancet holder, and a firing unit for driving the lancet holder and the lancet to move linearly. Thus, the user or the person under test can control the firing unit to drive the lancet forwards, causing the needle of the lancet to extend out of the front cap and to pierce the skin of the person under test to a certain depth and then to move back. During the lancing procedure, the person under test suffers pain due to vibration of the lancet in the procedure. More particularly, when the lancet reaches the maximum stroke in the lancing procedure, it vibrates severely, making the person under test feel miserable. Taking a close look at the reasons, vibration occurs due to unstable positioning of the lancet in the lancet holder and instability of the movement of the lancet holder. Improvements are desired.

### SUMMARY OF THE INVENTION

The present invention has been accomplished under the circumstances in view. It is the main object of the present invention to provide a lancing device, which is equipped with an improved lancet holder that holds the lancet firmly in position and moves the lancet smoothly with minimized vibration to reduce the sense of pain suffered by the user.

To achieve this and other objects of the present invention, a lancing device comprises a front cap detachably capped on a front side of a shell, a lancet comprising a body and a needle mounted in one end of the body, and a lancet holder linearly movably mounted in the shell and adapted for securing at least a part of the body of the lancet, wherein the lancet holder comprises at least one guide portion located at an outer perimeter thereof; the front cap comprises at least one guide track located at an inner wall thereof corresponding to the stroke of said at least one guide portion of the lancet holder; the at least one guide portion has the outer edge thereof configured in a substantially complementary relationship with the at least one guide track of the front cap.

Thus, when the lancing device uses the lancet to get a blood sample, the lancet will be held firmly by the lancet holder and moved stably with the lancet holder under the guidance of the guide track, minimizing vibration and reducing the sense of pain suffered by the user.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a schematic drawing illustrating a lancing device in accordance with a first embodiment of the present invention.

FIG. 2 is an exploded view of the lancing device in accordance with the first embodiment of the present invention.

FIG. 3 is a sectional view taken along line 3-3 of FIG. 2.

FIG. 4 is a sectional view taken along line 4-4 of FIG. 3.

FIG. 5 is a longitudinal sectional view of a lancing device in accordance with a second embodiment of the present invention.

FIG. 6 is a sectional view taken along line 6-6 of FIG. 5.

FIG. 7 is a longitudinal sectional view of a lancing device in accordance with a third embodiment of the present invention.

FIG. 8 is a sectional view taken along line 8-8 of FIG. 7.

FIG. 9 is a longitudinal sectional view of a lancing device in accordance with a fourth embodiment of the present invention.

### DETAILED DESCRIPTION OF THE INVENTION

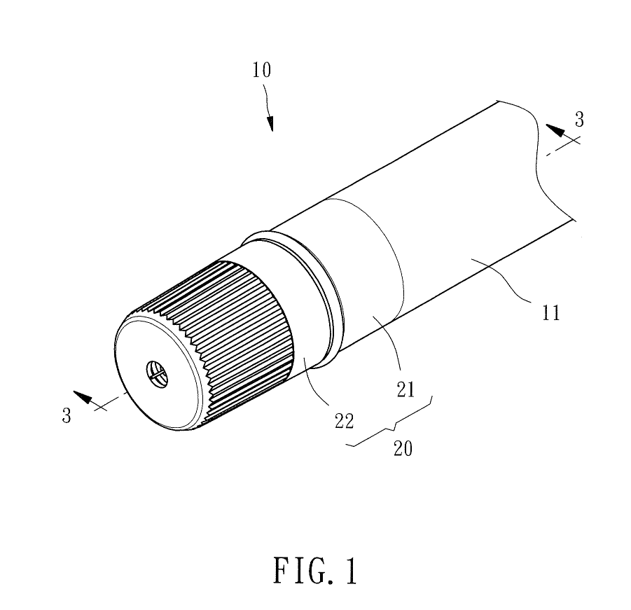

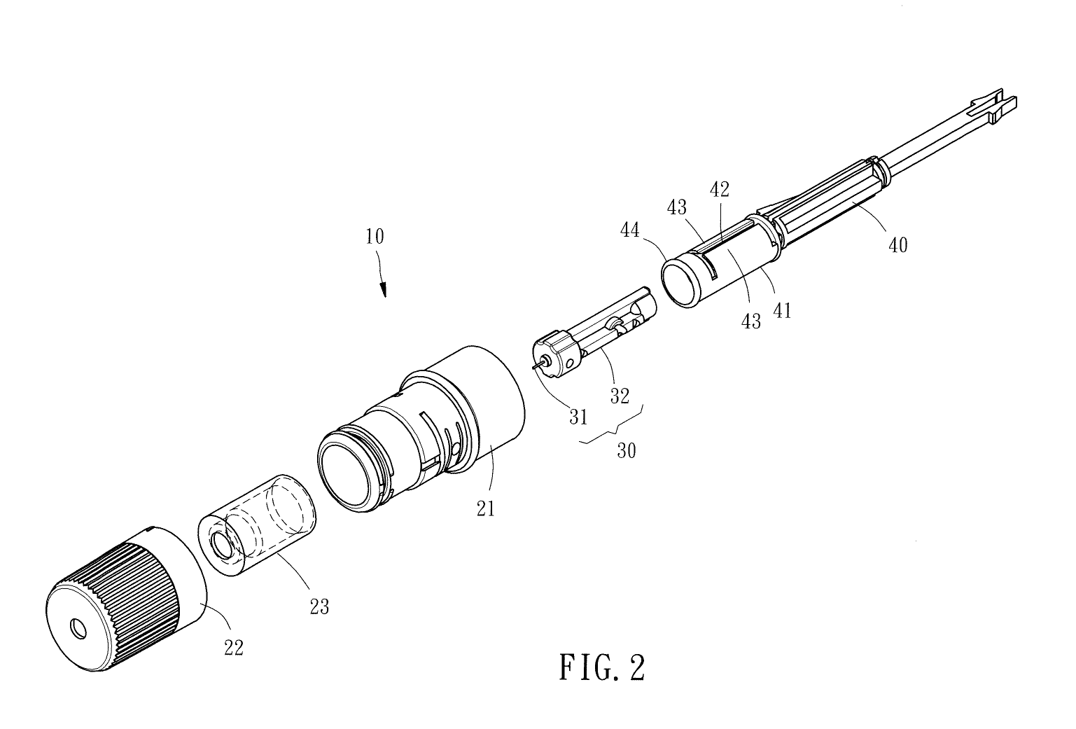

Referring to FIGS. 1 and 2, a lancing device 10 in accordance with a first embodiment of the present invention is shown. The lancing device 10 comprises a front cap 20, a lancet 30 and a lancet holder 40. The front cap 20 is detachably capped on a front end of a shell 11, comprising a tubular member 21, a cap member 22 capped on a front end of the tubular member 21 and a bushing 23 accommodated in the tubular member 21. The lancet 30 is mounted in a front end of the lancet holder 40. The lancet holder 40 is axially movably mounted in the shell 11, and drivable to move linearly by a firing unit (not shown). The structure of the firing unit is not the feature of the invention, therefore, no further detailed description in this regard will be necessary.

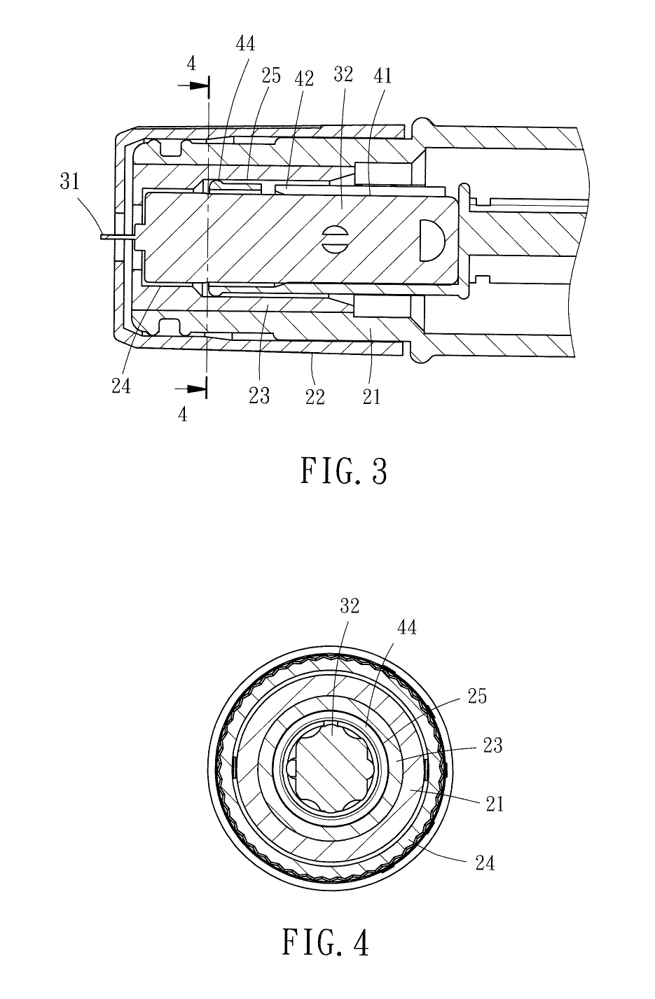

The lancet 30 is a component replaceable by the user, comprising a needle 31 and a body 32. The needle 31 is mounted at one end of the body 32. The lancet holder 40 in this embodiment is a one-piece member, comprising a barrel-like receptacle 41 located at a front end thereof for accommodating a major part or all of the body 32 of the lancet 30. The receptacle 41 ha an H-shaped cut 42 located on a middle part thereof, defining two clamping plates 43 for elastically clamping the body 32 of the lancet 30 to secure the lancet 30 positively in place. When the user operates the firing unit to drive the lancet 30 and the lancet holder 40, as shown in FIG. 3, the lancet 30 is moved through a longitudinal passage 24 in the bushing 23 to extend the needle 31 out of the cap member 22. Besides, the amount of the cut 42 may be one or more, and the shape of the cut 42 may be sawtooth-shaped or other form to provide elastic deformation.

Further, as shown in FIG. 2 and FIG. 3, the lancet holder 40 comprises at least one, for example, one guide portion 44 made in an annular shape and extending around the periphery of a front end thereof. The bushing 23 comprises at least one, for example, one guide track 25 defined in an inside wall thereof in a circular shape and adapted for mating with the guide portion 44. The outer edge of the guide portion 44 and the guide track 25 are substantially arranged in a complementary relationship, as shown in FIG. 4. In this embodiment, the guide portion 44 and the guide track 25 are circular in section, further, the distance between the guide portion 44 and the guide track 25 is in the range of 0.01˜0.3 mm.

It's worth mentioning that the design of the guide portion 44 and the guide track 25 is not limited to the aforesaid circular configuration. Alternatively, they can be designed to provide a triangular or rectangular configuration. Further, as an alternate form of the invention, the guide portion 44 can be mounted at any other location on the outer perimeter of the lancet holder 40, for example, at a front and/or rear part of the outer perimeter of the receptacle 41 of the lancet holder receptacle 40. Or, alternatively, the bushing 23 can be mounted at the cap member 22, or integrally made with the tubular member 21 to form a one-piece member. Alternatively, the lancet holder 40 can be formed of multiple components; the cut 42 of the lancet holder 40 can be configured in any other form.

Because of the characteristics that the lancet 30 is firmly mounted in the receptacle 41 of the lancet holder 40 and the lancet holder 40 can be accurately guided by the guide track 25 by means of the guide portion 44, when the user uses the lancing device 10, the lancet 30 and the lancet holder 40 can be moved smoothly with minimized vibration to reduce the sense of pain suffered by the user, or to achieve pain-free status.

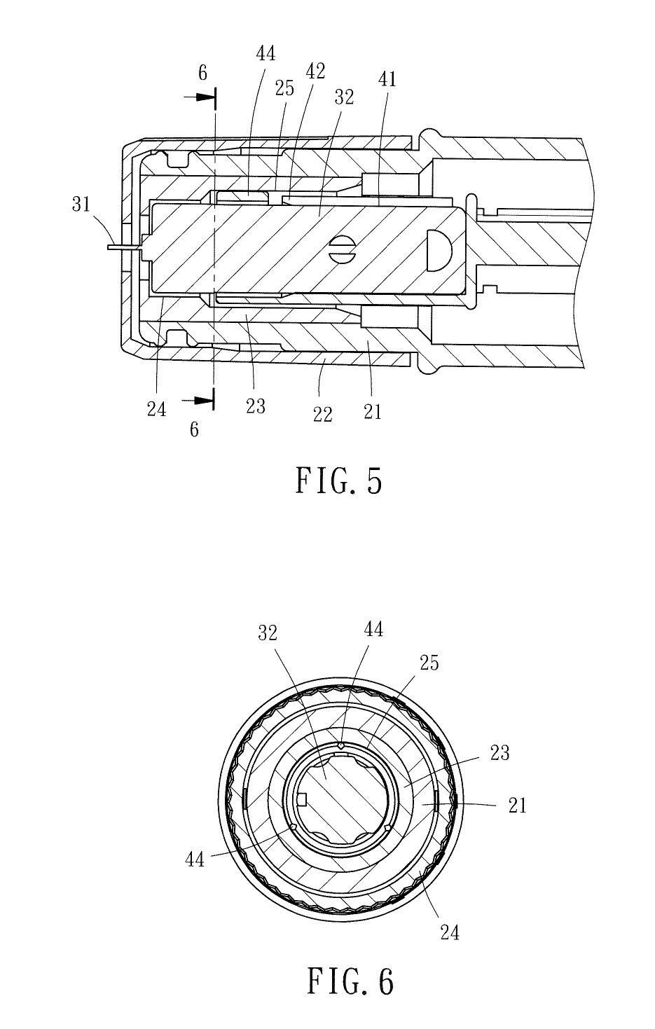

Referring to FIG. 5, a lancing device 10 in accordance with a second embodiment of the present invention is shown. This second embodiment is substantially similar to the aforesaid first embodiment with the exception that the number of the at least one guide portion 44 of the lancet holder 40 is 3, and these three guide portions 44 are longitudinal ribs equiangularly spaced around the outer perimeter of the receptacle 41. Because the outer edges of these guide portions 44 are substantially located on the same circumference, accurately controlling the distance between the diameter of the circumference and the inner diameter of the guide track 25 can maintain the guiding effect of the guide track 25.

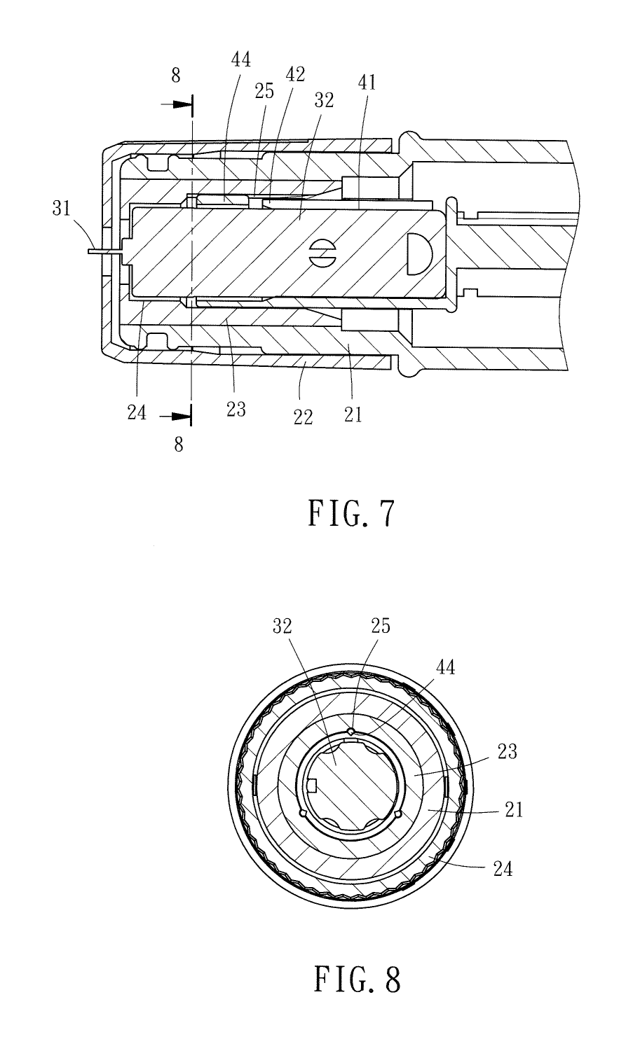

Referring to FIGS. 7 and 8, a lancing device 10 in accordance with a third embodiment of the present invention is shown. This third embodiment is substantially similar to the aforesaid second embodiment with the exception that the number of the at least one guide track 25 of the bushing 23 is 3, and these three guide tracks 35 are longitudinal grooves mating with the three guide portions 44 of the lancet holder 40.

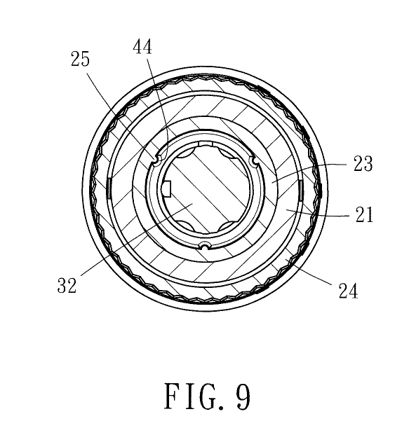

Referring to FIG. 9, a lancing device 10 in accordance with a fourth embodiment of the present invention is shown. This fourth embodiment is substantially similar to the aforesaid third embodiment with the exception that the raised and recessed configurations between the guide portions 44 and the guide tracks 25 are exchanged, i.e., the guide portions 44 are longitudinal grooves and the guide tracks 25 are longitudinal ribs mating with the longitudinal grooves to achieve the same guiding effect. More specifically, the number of the guide portions is not limited to 3; a different amount of guide portions is workable.

The design of the lancet holder guiding structure formed of the at least one guide portion of the lancet holder and the at least one guide track of the bushing effectively enhances the stability of the movement of the lancet holder. Further, because the lancet is firmly mounted in the lancet holder, vibration is minimized to reduce the sense of pain suffered by the user in the lancing operation.

1. A lancing device, comprising:
a front cap detachably capped on a front side of a shell;
a lancet comprising a body and a needle mounted in one end of said body; and
a lancet holder linearly movably mounted in said shell and adapted for securing at least a part of said body of said lancet;
wherein said lancet holder comprises at least one guide portion located at an outer perimeter thereof; said front cap comprises at least one guide track located at an inner wall thereof corresponding to the stroke of said at least one guide portion of said lancet holder, said at least one guide portion each having an outer edge thereof respectively configured in a substantially complementary relationship with said at least one guide track of said front cap.

2. The lancet holder as claimed in claim 1, wherein said lancet holder comprises a receptacle located at a front end thereof and configured for accommodating said body of said lancet, said receptacle comprising a at least one cut and two clamping plates defined by said cut for securing said body of said lancet.

3. The lancet holder as claimed in claim 2, wherein said cut is H-shaped or sawtooth-shaped.

4. The lancet holder as claimed in claim 1, wherein the number of said at least one guide portion and the number of said at least one guide track are 1, said guide portion having an annular shape, said guide track having a circular shape in section.

5. The lancet holder as claimed in claim 4, wherein said guide portion is extended around the periphery of a front end of said lancet holder.

6. The lancet holder as claimed in claim 1, wherein said at least one guide portion comprises a plurality of longitudinal ribs extending along the moving direction of said lancet holder.

7. The lancet holder as claimed in claim 6, wherein said at least one guide track comprises a plurality of longitudinal grooves respectively disposed corresponding to said longitudinal ribs of said at least one guide portion.

8. The lancet holder as claimed in claim 7, wherein the number of said at least one guide portion and the number of said at least one guide track are 3.

9. The lancet holder as claimed in claim 6, wherein said longitudinal ribs of said at least one guide portion have respective outer edges thereof substantially located on the same circumference; the number of said at least one guide track is 1, and this said guide track has a circular cross section.

10. The lancet holder as claimed in claim 1, wherein said at least one guide portion comprises a plurality of longitudinal grooves extending along the moving direction of said lancet holder; said at least one guide track comprises a plurality of longitudinal ribs respectively disposed corresponding to said longitudinal grooves.

11. The lancet holder as claimed in any claim 1, wherein said front cap comprises a tubular member, and a bushing mounted in said tubular member; said at least one guide track is located at an inside wall of said bushing.

12. The lancet holder as claimed in claim 11, wherein said bushing is integrated into said tubular member.

13. The lancet holder as claimed in claim 11, wherein said front cap further comprises a cap member capped on a front end of said tubular member.

14. The lancet holder as claimed in claim 1, wherein the distance between said at least one guide portion and said at least one guide track is in the range of 0.01-0.3 mm.
